(19) United States
(12) Patent Application Publication
(10) Pub. No.: US 2015/0352465 A1
(43) Pub. Date: Dec. 10, 2015

(54) Chromatography Media For Purifying Vaccines And Viruses

(71) Applicant: EMID MILLIPORE CORPORATION, Billerica, MA (US)

(72) Inventors: John Amara, Billerica, MA (US); Benjamin Cacace, Billerica, MA (US); David Yavorsky, Billerica, MA (US); John Boyle, Billerica, MA (US)

(21) Appl. No.: 14/759,426

(22) PCT Filed: Jan. 3, 2014
(86) PCT No.: PCT/US14/10158

§ 371 Date: Jul. 7, 2015

Related U.S. Application Data
(60) Provisional application No. 61/758,926, filed on Jan. 31, 2013.

(51) Int. Cl. B01D 15/36; C12N 7/00

(52) U.S. Cl. CPC B01D 15/361; B01D 15/362; B01D 15/363; C12N 7/00; C12N 2760/16051

(57) ABSTRACT

Adsorptive media for chromatography, particularly ionexchange chromatography, derived from a shaped fiber, useful for purifying viruses. In certain embodiments, the functionalized shaped fiber presents a fibrillated or ridged structure which greatly increases the surface area of the fibers when compared to ordinary fibers. Surface pendant functional groups can be added that provides ion-exchange functionality to the high surface area fibers. This pendant functionality is useful for the ion-exchange chromatographic purification of viruses, such as influenza.

This application claims priority of U.S. Provisional Application Ser. No. 61/758,926 filed Jan. 31, 2013, the disclosure of which is incorporated herein by reference.

### FIELD

The embodiments disclosed herein relate to chromatography media suitable for the purification of vaccines and viruses and for viral clearance applications for the purification of monoclonal antibody feed streams.

### BACKGROUND

The development of new purification technologies for the preparation of vaccines is of great interest, both as a response to recent pandemic outbreaks, as well as for emerging therapeutic applications. There is a general need for such new technologies in order to improve yields, increase product purity, and accelerate production rates. Currently employed vaccine purification technologies include cesium chloride density gradient centrifugation, tangential flow filtration, and chromatography. Each of these technologies provides distinct advantages and disadvantages and vaccine manufacturers must select the particular purification technology based on their production scale, purity, and product cost requirements. A typical vaccine purification process is described in the process flow diagram set forth in FIG. 1.

Both tangential flow filtration and gradient centrifugation processes are widely used in the production of vaccines, but these unit operations are expensive and time-consuming batch operations, are poorly-scalable, require specialized equipment and personnel, and provide low yields and loss of infectivity. The equipment used for such operations is hardly disposable and expensive regeneration, cleaning, and validation processes must be performed in order to prepare the purification equipment for the next batch.

In contrast, the use of bead based resins for bind/elute chromatographic purification of vaccines is of interest since the purification processes can be performed at much larger scales. Unfortunately, commercially available resins for these applications typically present pore sizes that are much too small to be accessed by the larger virus particles. As a result, such media demonstrate low binding capacity since the viruses can only access the external surfaces of the beads. The low binding capacity, coupled with the high costs associated with chromatography resins suitable for this application, requires manufacturers to perform numerous bind/elute and column regeneration cycles using the chromatography media in order to make such processes cost-effective. The regeneration processes further increase production costs due to decreased product throughput, increased consumption of buffers and cleaning agents, validation costs, and increased capital equipment requirements. Emerging technologies are currently in development that may provide increased binding capacities for viruses and these include membrane adsorbers, monoliths, and flow-through adsorber purification methods using commercial resin systems. While membrane adsorbers and monoliths may enable increased binding capacities for these applications, these technologies typically have their own scale limitations and the extremely high cost of such purification media precludes the use of these products as disposable devices and may further limit their adoption into a traditionally price-sensitive vaccine industry.

### SUMMARY

In order to address many of the limitations of the purification technologies currently known in the art, a new type of chromatography media has been developed that comprises a very low-cost thermoplastic fiber and ligand functionality on the surface of the fiber. The ligand is capable of selectively binding viruses from a cell culture feed stream, such as by ion-exchange. The bound virus can be subsequently released from the chromatography media upon a change in the solution conditions, for example, through the use of an elution buffer with a higher ionic strength. The fiber-based stationary phase is non porous and displays a convoluted surface structure that provides a sufficient surface area for high virus binding capacity. Since the virus binding occurs only on the surface of the fiber, there are no size exclusion issues with virus binding as is seen in the case of porous bead-based bind/elute systems. Furthermore, since the virus particles can be transported directly to the ligand site by convection, there are no diffusion limitations in the system and the vaccine feed stream, for example, may be processed at much higher flow rates or shorter residence times.

In accordance with certain embodiments, the chromatography media is derived from a shaped fiber. In certain embodiments, the shaped fiber presents a fibrillated or ridged structure (e.g., FIG. 1(b)). These ridges can greatly increase the surface area of the fibers when compared to ordinary fibers (e.g., FIG. 1(a)). Thus, high surface area is obtained without reducing fiber diameter, which typically results in a significant decrease in bed permeability and a corresponding reduction in flow rate. An example of the high surface area fiber in accordance with certain embodiments is “winged” fibers, commercially available from Allasso Industries, Inc. (Raleigh, N.C.). A cross-sectional SEM image of an Allasso winged fiber is provided in FIG. 1(d). These fibers present a surface area in the range of approximately 1 to 14 square meters per gram. Surface area measurement of the fiber media is determined by conventional gas adsorption techniques such as the method of Brunauer, Emmett, and Teller (BET) using krypton or nitrogen gases.

Also disclosed herein is a method to add surface pendant functional groups that provides anion-exchange (AEX) functionality, for example, to the high surface area fibers. This pendant functionality is useful for the ion-exchange chromatographic purification of vaccines and viruses, such as influenza.

Embodiments disclosed herein also relate to methods for purification of vaccines and viruses with media comprising a high surface area functionalized fiber. These methods can be carried out in a flow through mode or a bind/elute mode.

In accordance with certain embodiments, the media disclosed herein have high bed permeability (e.g., 300-900 mDarcy), low material cost relative to bead-based chromatographic media, 20-mg/mL BSA dynamic binding, high separation efficiencies (e.g., HETP <0.1 cm), 50-200 mg/g IgG static binding capacity, and fast convective dominated transport of adsorbate to ligand binding sites.

In accordance with certain embodiments, the use of unique, high surface area, extruded fibers (e.g., thermoplastic fibers) allows for high flow permeability (liquid) and uniform flow distribution when configured as a packed bed of randomly oriented cut fibers of lengths between 0.5-6 mm. Chemical treatment methods to functionalize such fiber surfaces are provided to enable separations based on adsorptive interaction(s). Chemical treatment methods can impart a variety of surface chemical functionalities to such fibers based on either ionic, affinity, hydrophobic, etc. interactions or combinations of interactions. The combined economies of fiber production and simple surface chemical treatment processes yield an economical and readily scalable technology for purification operations in biopharmaceutical as well as vaccine production and virus purification.

In accordance with certain embodiments, an adsorptive separations material is provided that allows for fast processing rates, since mass transport for solutes to and from the fiber surface is largely controlled by fluid convection through the media in contrast to bead-based media where diffusional transport dictates longer contact times and therefore slower processing rates. The ability to capture or remove large biological species such as viruses is provided, which cannot be efficiently separated using conventional bead-based media due to the steric restrictions of bead pores.

### BRIEF DESCRIPTION OF THE DRAWINGS

FIG. 1(a) is a schematic view of a fiber in accordance with the prior art;

FIG. 1(b) is a schematic view of a ridged fiber that can be used in accordance with certain embodiments;

FIG. 1(c) is a schematic view of the fiber of FIG. 1b with attached pendant groups in accordance with certain embodiments;

FIG. 1(d) is an SEM image of a ridged fiber that can be used in accordance with certain embodiments;

FIG. 1(e) is a schematic view of functionalization of fibers in accordance with certain embodiments;

FIG. 2 is a plot of the static binding capacity of BSA-latex particles for selected adsorbants in accordance with certain embodiments;

FIGS. 3(a)-(d) are SEM images of various fibers;

FIG. 4 is a plot of Φ6 LRV for flow through fractions collected for AEX fiber media, as well as selected commercial membrane adsorbers and a bead-based AEX media;

FIG. 5 is a plot of elution pool Φ6 titers;

FIG. 6 is a plot of viral clearance comparisons;

FIG. 7 is a plot of influenza breakthroughs;

FIG. 8 is a plot of influenza breakthroughs;

FIG. 9 is a plot of flow through MVM clearance LRV values;

FIG. 10 is a cross-sectional view of a fiber in the shape of a snow flake in accordance with certain embodiments;

FIG. 11 is a cross-sectional view of a fiber in the shape of a sun in accordance with certain embodiments;

FIG. 12 is a cross-sectional view of a fiber in the shape of a daisy in accordance with certain embodiments;

FIGS. 13(a)-(e) are cross-sectional views of fibers with projections and branched sub-projections in accordance with certain embodiments; and

FIGS. 14(a)-(d) are cross-sectional views of shaped fibers with increased surface area in accordance with certain embodiments.

### DETAILED DESCRIPTION

The shaped fiber medium in accordance with the embodiments disclosed herein relies only on the surface of the fiber itself. Since the shaped fiber affords high surface area as well as high permeability to flow, the addition of an agarose hydrogel or porous particulates are not necessary to boost the available surface area on the fiber support to meet performance objectives with respect to capacity and efficiency. Moreover, without the need to enhance surface area by the addition of a hydrogel or porous particulate, the manufacturing cost of the media described herein is kept to a minimum.

Fibers may be of any length and diameter and are preferably cut or staple fibers or a non-woven fabric. They need not be bonded together as an integrated structure but can serve effectively as individual discrete entities. They may be in the form of a continuous length such as thread or monofilament of indeterminate length or they may be formed into shorter individual fibers such as by chopping fibrous materials (e.g., staple fibers) such as non-woven or woven fabrics, cutting the continuous length fiber into individual pieces, formed by a crystalline growth method and the like. Preferably the fibers are made of a thermoplastic polymer, such as polypropylene, polyester, polyethylene, polyamide, thermoplastic urethanes, copolyesters, or liquid crystalline polymers. Fibers with deniers of from about 1-3 are preferred. In certain embodiments, the fiber has a cross-sectional length of from about 1 μm to about 100 μm and a cross-sectional width of from about 1 μm to about 100 μm. One suitable fiber has a cross-sectional length of about 20 μm and a cross-sectional width of about 10 μm, and a denier of about 1.5. Fibers with surface areas ranging from about 10,000 cm²/g to about 1,000,000 cm²/g are suitable. Preferably the fibers have a cross-sectional length of about 10-20 μm.

In certain embodiments, the fibers can readily be packed under compression into a device or container with appropriate ports and dimensions to suit the applications described. The fibers also can be used in a pre-formed bed format such as nonwoven sheetstock material created by a spunbond (continuous filament) or wet-laid (cut fiber) process, common in the nonwovens industry. Suitable pre-formed fiber formats include sheets, mats, webs, monoliths, etc.

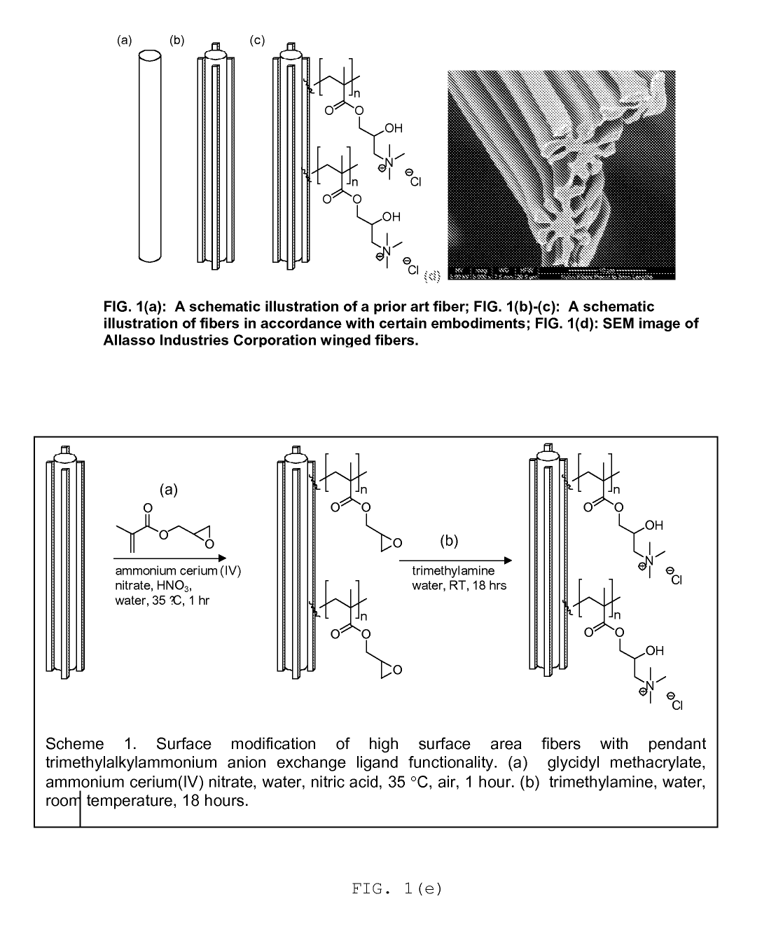

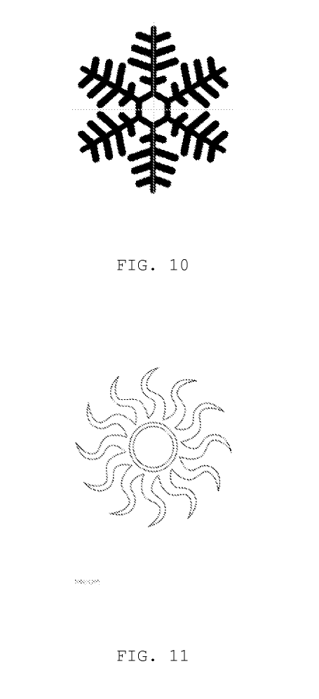

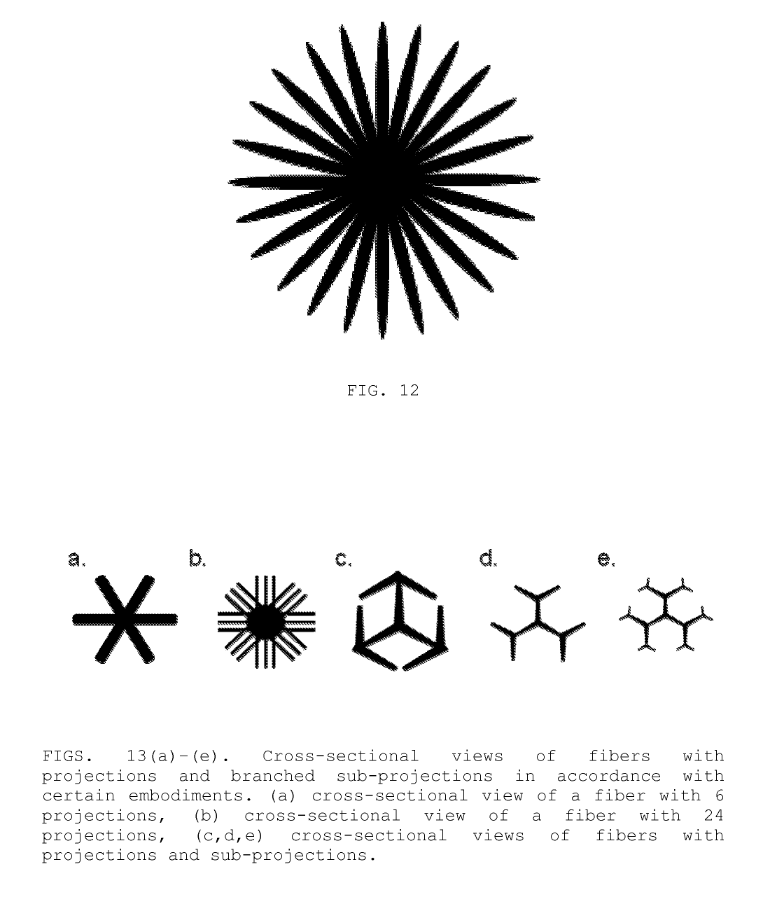

In certain embodiments, the fiber cross-section is generally winged-shaped, with a body region, and a plurality of projections extending radially outwardly from the body region. The projections form an array of co-linear channels that extend along the length of the fiber, typically 20-30 such channels per fiber. In certain embodiments, the length of the projections is shorter than the length of the body region. In certain embodiments, the fiber cross-section is generally winged-shaped, with a middle region comprising a longitudinal axis that runs down the center of the fiber and having a plurality of projections that extend from the middle region (FIG. 1(d)). In certain embodiments, a plurality of the projections extends generally radially from the middle region. As a result of this configuration, a plurality of channels is defined by the projections. Suitable channel widths between projections range from about 200 to about 1000 nanometers. Suitable fibers are disclosed in U.S. Patent Publication No. 2008/0105612, the disclosure of which is incorporated herein by reference. In certain embodiments, the fiber includes a body region and one or more projections extending from the body region. The projections also can have projections extending from them. The projections can be straight or curved. The projections can be of the substantially same length, or of different lengths. The body region can have regions of thickness greater than the thickness of the projections. Exemplary shapes include a snowflake shape as shown in FIG. 10, a sun shape as shown in FIG. 11, and a daisy shape as shown in FIG. 12. More specifically, the snowflake shape in FIG. 10 includes a central body portion with a plurality of projections extending outwardly therefrom. Each of these projections has a plurality of shorter secondary or sub projections of varying lengths extending outwardly from it along its length. The sun shape shown in FIG. 11 also includes a central body portion, and has a plurality of curved projections extending outwardly therefrom. The daisy shape shown in FIG. 12 includes a central solid body portion, with a plurality of projections extending outwardly therefrom, these projections being devoid of additional projections.

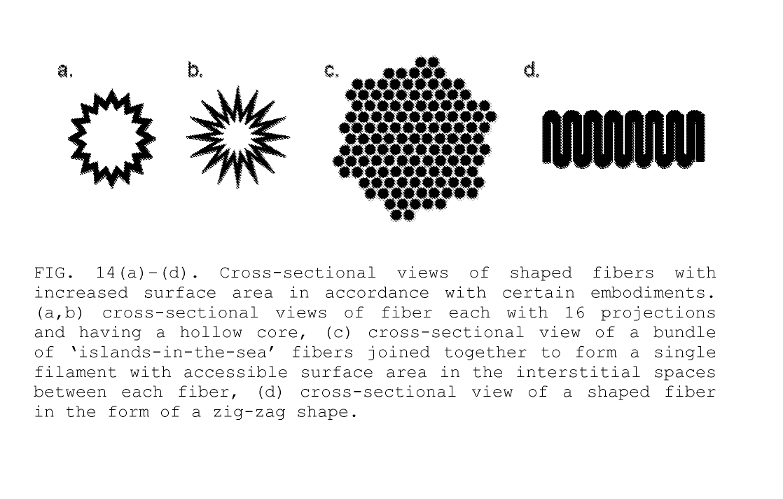

The body region can be solid (e.g. FIG. 12) or hollow (e.g. FIGS. 10 and 11), substantially linear or non-linear. Other exemplary shapes include shaped fibers comprising branched structures as shown in FIG. 13(a)-(e). Thus, FIG. 13(a) is a star shape, with a solid central body region and six straight equally paced projections extending outwardly therefrom in a symmetrical pattern. The fiber shown in FIG. 13(b) has a solid central body region, with sets of straight projections extending outwardly therefrom, each projection within a set extending in the same direction. The fiber shown in FIG. 13(c) has a central body region with three straight equally spaced projections extending therefrom in different directions. Each projection has its terminal free end secondary or sub projections extending therefrom at an angle towards the central body region. The fiber in FIG. 13(d) is similar to that of FIG. 13(c), except that the secondary or sub projections extend at an angle away from the central body region. The fiber in FIG. 13(d) is similar to that of FIG. 13(d), except that each secondary or sub projection has additional projections at its terminal free end.

Other exemplary shapes include fibers with hollow cores, bundled microfilaments, or fibers in the shape of wavy ribbons, as shown in FIG. 14(a)-(d). FIGS. 14(a) and (b) illustrate closed polygons with hollow cores and a plurality of projections defining alternating peaks and valleys. FIG. 14(c) illustrates a bundle of fibers joined together in a cluster to form a single filament with accessible surface area in the interstitial spaces between each fiber. FIG. 14(d) illustrates a shaped fiber having a zig-zag pattern.

The fiber shapes may be produced using a bi-component fiber spinning machine from Hills, Inc. (West Melbourne, Fla.). Shaped bi-component fibers can be prepared using commercially available fiber spinning equipment and custom-designed fiber die stacks as described in U.S. Pat. No. 5,162,074, the disclosure of which is incorporated herein by reference. Two extruders feed melt processable materials into a common spin head. The spin head contains a die stack that splits and redirects the melt flow into separate filaments which are collected after exiting through a spinneret. The cross section of each filament has the desired fiber shape in the primary material and a secondary material acting as a negative to the desired fiber shape. The presence of the secondary material allows fiber features in the fiber cross section that would be impossible if the primary material were extruded alone both in terms of feature size and proximity. After extrusion, the secondary material is removed, usually by dissolution, leaving the high surface area fiber with the desired cross section. The details of the final cross section of the fiber is determined by a combination of die stack, processing conditions, spinneret shape, and choice of primary and secondary polymers.

The die stack can be made to produce a variety of very intricate, complicated cross sections. The primary material can be any material that can be melt spun: polypropylene, polyester, polyamide, polyethylene, etc. The secondary material could also be any melt spinnable material; however it is preferred the secondary material is easily removed so the preferred materials are soluble polymers such as: polylactic acid, polyvinyl alcohol, soluble copolyesters, etc.

In accordance with certain embodiments, surface pendant functional groups are installed that provide an anion-exchange functionality to the high surface area fibers. This pendant functionality is useful for the anion-exchange chromatographic purification of vaccines and viruses such as influenza.

The surface functionalization of the high surface area fibers can be accomplished by a two-step process. A suitable functionalization process is grafting polymerization, and is exemplified in Scheme 1 shown in FIG. 1(e). In this embodiment, the high surface area fibers are reacted with an aqueous solution of glycidyl methacrylate monomer, ammonium cerium(IV) nitrate, and HNO₃ at 35° C. in air for 1 hour. Under these conditions, cerium oxidation of the nylon fiber surface generates free radicals and initiates a surface grafting polymerization of the glycidyl methacrylate polymer. Under such conditions, the surface initiated polymerization process produces a polymeric “tentacle” of polymerized glycidyl methacrylate monomer. In this way, the glycidyl methacrylate polymer is covalently attached to the fiber surface. Such processes are known as grafting polymerizations.

In the second synthetic step, in certain embodiments the poly(glycidyl methacrylate) modified fiber material is quickly washed with water and treated with an aqueous solution of trimethylamine (25 wt %) at room temperature for 18 hours. Under these conditions, any residual epoxy groups on the poly(glycidyl methacrylate) tentacles may react with the trimethylamine, affording a pendant cationic trimethylalkylammonium (Q) functionality that can provide the desired anion exchange functionality for vaccine purification applications.

A suitable column packing density of between about 0.1-0.4 g/ml, preferably about 0.32 g/ml, at a bed height of 1-5 cm will provide sufficient flow uniformity for acceptable performance in a chromatographic evaluation.

In certain embodiments, the media (functionalized packed fibers) may be delivered to the user in a dry, prepacked format, unlike bead-based media. The fibers can be fused either by thermal or chemical means to form a semi-rigid structure that can be housed in a pressure vessel. By such a construction, the media and accompanying device can be made ready-to-use. Chromatographic bead-based media is generally delivered as loose material (wet) wherein the user is required is load a pressure vessel (column) and by various means create a well-packed bed without voids or channels. Follow-up testing is generally required to ensure uniformity of packing. In contrast, in accordance with certain embodiments, no packing is required by the user as the product arrives ready for service.

The shaped fiber media offers certain advantages over porous chromatographic beads by nature of its morphology. Typically in bead-based chromatography, the rate limiting step in the separation process is penetration of the adsorbate (solute) into the depths of porous beads as controlled by diffusion; for macromolecules such as proteins, this diffusional transport can be relatively slow. For the high surface area fibers disclosed herein, the binding sites are exposed on the exterior of the fibers and therefore easily accessed by adsorbate molecules in the flow stream. The rapid transport offered by this approach allows for short residence time (high flow velocity), thereby enabling rapid cycling of the media by means such as simulated moving bed systems. As speed of processing is a critical parameter in the production of biologics, fiber-based chromatographic media as described herein has particular process advantages over conventional bead-based media.

Conventional chromatographic resins start with porous beads, typically of agarose, synthetic polymer, and silica or glass. These materials are generally of high cost: unfunctionalized agarose beads can cost between $300-$350 per liter and controlled pore glass between $600-$1000 per liter. By contrast, a nonwoven bed of high surface area fibers as described herein in the appropriate densities and thickness to achieve good chromatographic properties are estimated to cost between $20-$50 per liter. This cost advantage will raise the likelihood that this new chromatographic media can be marketed as a “disposable” technology (e.g., single use) suitably priced for use and disposable after single use or most likely after multiple cycles within one production campaign.

The surface functionalized fiber media of the embodiments disclosed in U.S. Patent Publication No. 2012/0029176 the disclosure of which is incorporated herein by reference (e.g., SP functionalized Allasso fibers, SPF1) demonstrates a high permeability in a packed bed format. Depending on the packing density, the bed permeability can range from >14000 mDarcy to less than 1000 mDarcy. At low packing density of 0.1 g/mL (1 g media/9.3 mL column volume), a bed permeability of 14200 mDarcy at a linear velocity of 900 cm/hr was measured. This value does not change over a wide velocity range (400-1300 cm/hr). Such behavior indicates that the packed fiber bed does not compress at high linear velocity. Subsequent compression of the surface functionalized fiber media (SP functionalized Allasso fibers, SPF1) to a higher packing density of 0.33 g/mL (1 g media/2.85 mL column volume), afforded a bed permeability of 1000 mDarcy at a linear velocity of 900 cm/hr. Likewise, this value of 1000 mDarcy was unchanged over a linear velocity range of 400-1300 cm/hr. Suitable packing densities include between about 0.1 and about 0.5 g/ml.

For a conventional packed-bed, ion exchange chromatography media employed for bioseparations, such as ProRes-S (Millipore Corp, Billerica, Mass.), permeability values of 1900 mDarcy were measured for a packed bed of similar dimensions to the case above (3 cm bed depth, 11 mm ID Vantage column, 2.85 mL column volume). For membrane adsorbers, typical permeability values are in the range of 1-10 mDarcy. For ProRes-S, no significant change in bed permeability was measured over a range of velocities from 400-1300 cm/hr. While this behavior was expected for a semi-rigid bead, such as ProRes-S; a more compressible media (ex. agarose beads) is expected to demonstrate significant decreases in bed permeability at high linear velocities (>200 cm/hr) due to significant compression of the packed bed.

Examples of the high surface area fiber surface functionalization and trimethylamine epoxy ring opening procedures are provided below (Examples 1 and 2).

### Preparation of Trimethylalkylammonium (Q) Tentacle Functionalized High Surface Area Fibers (AEX Fiber Media)

### Example 1

### Graft Polymerization of Un-Modified Nylon Fibers

Into a 500 mL bottle were added 10 g of Allasso nylon fibers and water (466 mL). 1 M HNO₃ solution (14.4 mL, 14.4 mmol) were added to the reaction mixture, followed by addition of a 0.4 M solution of ammonium cerium(IV) nitrate in 1 M HNO₃ (1.20 mL, 0.480 mmol). The reaction mixture was agitated for 15 minutes. Glycidyl methacrylate (GMA, 3.39 g, 24 mmol) was added and the reaction mixture was heated to 35° C. for 1 hour. After cooling to room temperature, the solids were washed with DI water (3×300 mL) and the damp material was used immediately in the following step.

### Example 2

### Q-Functionalization of Epoxy-Functionalized Fibers (AEX Fiber Media)

Into a 2 L bottle were added the damp GMA functionalized fibers from example 1 above, water (500 mL) and a solution of 50 wt % trimethylamine (aq.) in methanol (500 mL). The mixture was agitated at room temperature for 18 hours. The fiber solids were subsequently washed with a solution of 0.2 M ascorbic acid in 0.5 M sulfuric acid (3×400 mL), DI water (3×400 mL), 1 M sodium hydroxide solution (3×400 mL), DI water (3×400 mL) and ethanol (1×400 mL). The material was placed in an oven to dry at 40° C. for 48 hrs. Obtained 11.74 g of a white fibrous solid.

Functional Performance of the AEX Fiber Media.

The performance of the AEX fiber media described in Example 2 was evaluated for various viral clearance and vaccine purification applications as described in the examples shown below.

### Example 3

### AEX Fiber Media Column Packing

Into an 11 mm ID Vantage column were added a slurry of 1.0 g of the AEX fiber media described in Example 2 above in 100 mL of 25 mM tris buffer (pH 8). The fiber media was compressed to a bed depth of 3.0 cm (2.85 mL column volume, 0.35 g/mL fiber packing density). Fiber bed permeability was assessed by flowing 25 mM Tris pH 8 buffer through the column at a flow rate of 2.0 mL/min and measuring the column pressure drop by means of an electronic pressure transducer. Fiber bed permeability values are provided in Table 1 below.

TABLE 1
Fiber media column packing

| | | | | Pressure, PSI | | Permeability, | |
|---|---|---|---|---|---|---|---|
| | Media type | Bed Depth, cm | | (flowrate, | | mDarcy | |
| Column Type | (amt) | (CV, mL), | | mL/min) | | (velocity, cm/hr) | |
| 11 mm Vantage | AEX fibers ex. | 3.0 | cm | 6.5 | PSI | 724 | mDa |
| | 2 (1.0 g) | (2.85 | mL) | (6.1 | mL/min) | (384 | cm/hr) |
| 11 mm Vantage | AEX fibers ex. | 3.0 | cm | 13 | PSI | 722 | mDa |
| | 2 (1.0 g) | (2.85 | mL) | (12 | mL/min) | (778 | cm/hr) |

### Example 4

### Simulation of Virus Binding to the AEX Fiber Media Using BSA-Coated Latex Beads

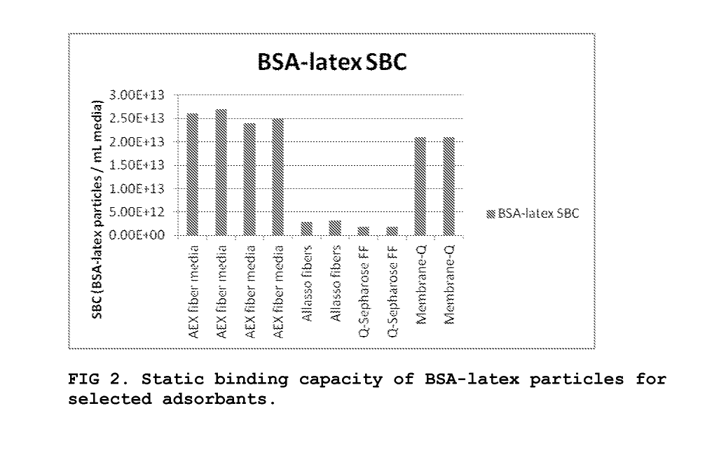

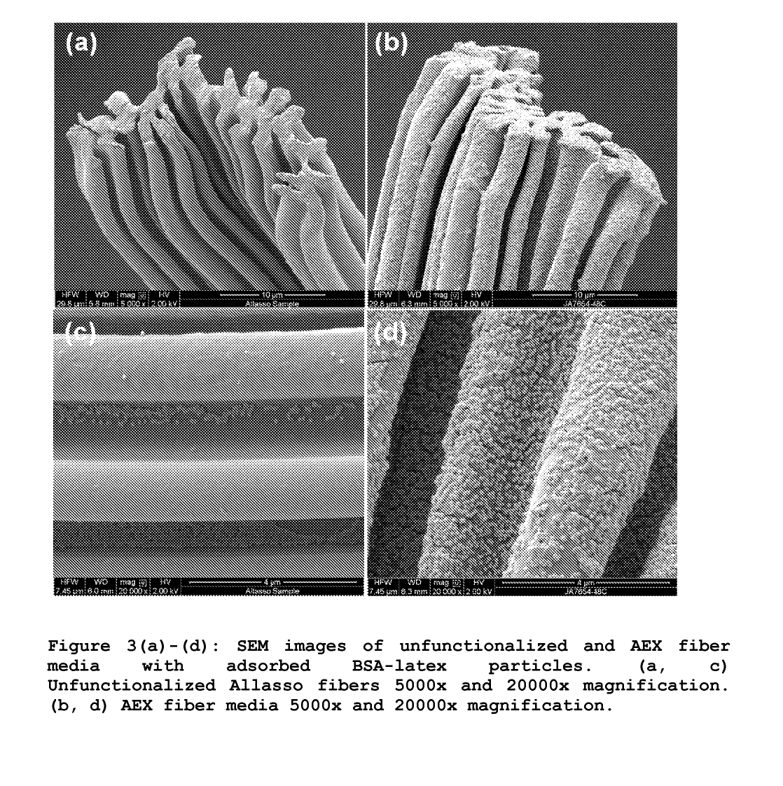

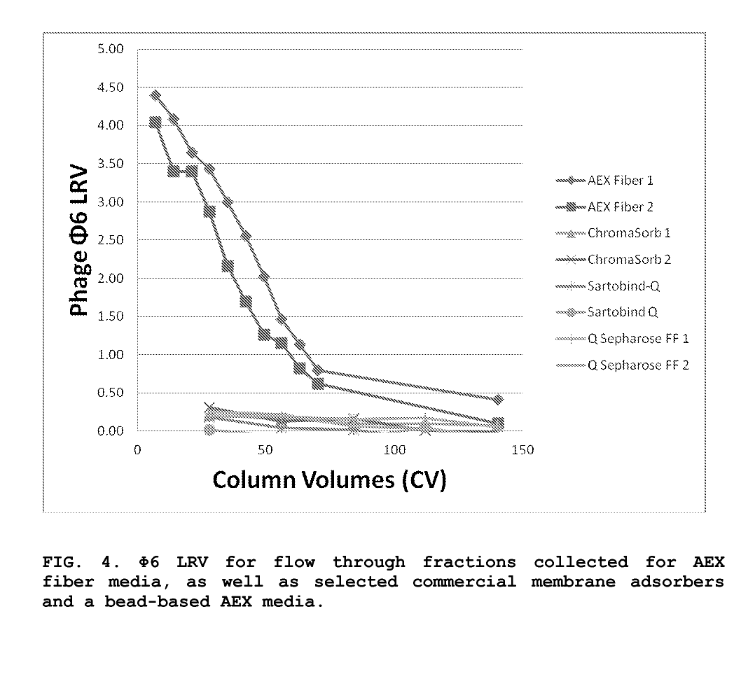

BSA-coated polystyrene latex particles (100 nm particle diameter) from Postnova Analytics Inc. were used as a model to simulate the size and charge characteristics of the influenza virus. A 2 mg/mL solution of the BSA-latex particles was prepared in 25 mM Tris buffer at pH and the static binding capacity of the AEX fiber media was determined and compared to that of a commercial Q-type resin (Q-Sepharose Fast Flow, GE Healthcare Life Sciences Inc.) as well as that of a commercial Q-type membrane adsorber (Membrane-Q). These results are summarized in Table 2 below and FIG. 2. The AEX fiber media has a significantly greater static binding capacity for the BSA coated latex particles than either the unfunctionalized Allasso fiber media or the commercial Q-Sepharose Fast Flow chromatography resin. The low binding capacity of the Q-Sepharose resin may be explained by the limited available surface area that is accessible by the large BSA-latex particles. Furthermore, the binding capacity for the AEX fiber media is comparable to that of the commercial Membrane-Q membrane adsorber. FIG. 3 provides SEM images of the AEX fiber media and the unmodified Allasso winged fibers after the static binding experiment using the BSA-latex particles. For the AEX fiber media, a significant quantity of the particles is observed nearly completely covering the fiber surface. In the case of a control experiment using the unfunctionalized Allasso fibers, only very few particles are adsorbed to the surface of the untreated Allasso fiber. In this case, any binding may be attributed to non-specific binding interactions between the BSA-latex particles and the untreated Allasso fiber.

TABLE 2
BSA-latex SBC for selected media

| | | BSA-latex | Final BSA-latex | Static binding |
|---|---|---|---|---|
| | | solution volume, | solution | capacity (BSA |
| | | mL (# of | concentration | particles/mL |
| Sample ID | Media amt, mL | particles) | (particles/mL) | media) |
| AEX fiber media | 0.10 mL¹ | 1.0 mL | 1.40 × 10¹² | 2.6 × 10¹³ |
| | | (4.09 × 10¹²) | | |
| AEX fiber media | 0.11 mL | 1.0 mL | 1.26 × 10¹² | 2.7 × 10¹³ |
| | | (4.09 × 10¹²) | | |
| AEX fiber media | 0.10 mL | 1.0 mL | 1.36 × 10¹² | 2.4 × 10¹³ |
| | | (3.85 × 10¹²) | | |
| AEX fiber media | 0.11 mL | 1.0 mL | 1.33 × 10¹² | 2.5 × 10¹³ |
| | | (3.85 × 10¹²) | | |
| Allasso fibers | 0.10 mL | 1.0 mL | 3.80 × 10¹² | 2.9 × 10¹² |
| | | (4.09 × 10¹²) | | |
| Allasso fibers | 0.10 mL | 1.0 mL | 3.78 × 10¹² | 3.2 × 10¹² |
| | | (4.09 × 10¹²) | | |
| Q-Sepharose FF | 1.0 mL | 1.0 mL | 2.30 × 10¹² | 1.8 × 10¹² |
| | | (4.09 × 10¹²) | | |
| Q-Sepharose FF | 1.0 mL | 1.0 mL | 2.32 × 10¹² | 1.8 × 10¹² |
| | | (4.09 × 10¹²) | | |
| Membrane-Q | 0.14 mL | 1.0 mL | 8.96 × 10¹¹ | 2.1 × 10¹³ |
| | | (3.85 × 10¹²) | | |
| Membrane-Q | 0.14 mL | 1.0 mL | 9.36 × 10¹¹ | 2.1 × 10¹³ |
| | | (3.85 × 10¹²) | | |

¹Fiber media volume based on a 0.35 g/mL fiber packing density

### Example 5

### Fiber Media Capability for the Bind/Elute Purification of Viruses

The results of static binding capacity and elution recovery measurements for bacteriophage Φ6 are provided in Table 3 below. Into 5 plastic centrifuge tubes were added the AEX fiber media of Example 2 and unfunctionalized Allasso fiber samples in the amounts described in the Table below. Each of the fiber samples and the control tube were equilibrated with 5 mL of 25 mM Tris buffer (pH 8, with 0.18 mg/mL HSA) with agitation for 10 minutes. The tubes were spun at room temperature in a table top centrifuge at 4000 rpm for 10 minutes to pellet the fiber media. 2.5 mL of the supernatant was removed and 2.5 mL of a 1.7×10⁷ pfu/mL Φ6 solution in 25 mM Tris buffer (pH 8, with 0.18 mg/mL HSA) were added to each tube. The samples were agitated at room temperature for 1 hour. Afterwards, the tubes were spun at room temperature in a table top centrifuge at 4000 rpm for 15 minutes to pellet the fiber media. 2.5 mL of the supernatant was removed and these samples were assayed for unbound Φ6 by plaque-forming assay. The tubes were washed 3 times with 2.5 mL washings of 25 mM Tris buffer (pH 8, with 0.18 mg/mL HSA) with centrifugation to pellet the fiber media in between each wash and removal of 2.5 mL of the supernatant. After washing, 2.5 mL of a 1.0 M NaCl solution in 25 mM Tris buffer (pH 8, with 0.18 mg/mL HSA) were added to each tube (5 mL total volume, final NaCl concentration is 0.5 M). The samples were agitated at room temperature for 10 minutes. Afterwards, the tubes were spun at room temperature in a table top centrifuge at 4000 rpm for 10 minutes to pellet the fiber media. 2.5 mL of the supernatant was removed and these elution samples were assayed for eluted Φ6 by plaque forming assay. The Q-functionalized tentacle fiber media of Example 2 demonstrates a significant bacteriophage Φ6 log reduction value (LRV) of 3.1 and an elution recovery yield of 40%. This performance is comparable to membrane-based anion-exchange media employed in commercial viral chromatography applications. The Q-functionalized fiber media of the present invention can be integrated into a pre-packed device format or a chromatography column for flow-through viral clearance or bind/elute viral purification applications. In contrast, the unfunctionalized Allasso fiber samples show no appreciable binding capacity for Φ6 bacteriophage (Φ6 LRV=0).

TABLE 3
Static binding capacity measurement. Challenge: 2.5 mL of
1.7E7 pfu/mL bacteriophage Phi6 in 25 mM Tris (pH 8) with
0.18 mg/mL HSA. Elution buffer: 0.5M NaCl in 25 mM Tris
(pH 8) with 0.18 mg/ml HSA.

| | | | | Elution | |
|---|---|---|---|---|---|
| | | | Φ6 | Φ6 | |
| | | Φ6 titer | bound | titer | % recovery, |
| Sample | Amt (g) | (pfu/mL) | (LRV) | (pfu/mL) | Φ6 |
| Control tube | — | 2.10 × 10⁷ | — | 2.15 × 10⁶ | — |
| Example 2 | 0.051 g | 1.39 × 10⁴ | 3.18 | 8.45 × 10⁶ | 40.3% |
| Example 2 | 0.052 g | 1.65 × 10⁴ | 3.10 | 8.15 × 10⁶ | 38.8% |
| Allasso non- | 0.051 g | 2.09 × 10⁷ | 0.00 | 8.65 × 10⁵ | — |
| functionalized | | | | | |
| fibers | | | | | |
| Allasso non- | 0.050 g | 2.32 × 10⁷ | −0.04 | 7.10 × 10⁵ | — |
| functionalized | | | | | |
| fibers | | | | | |

### Example 6

### Determination of Φ6 LRV, Φ6 Binding Capacity, and Elution Pool Φ6 Recovery

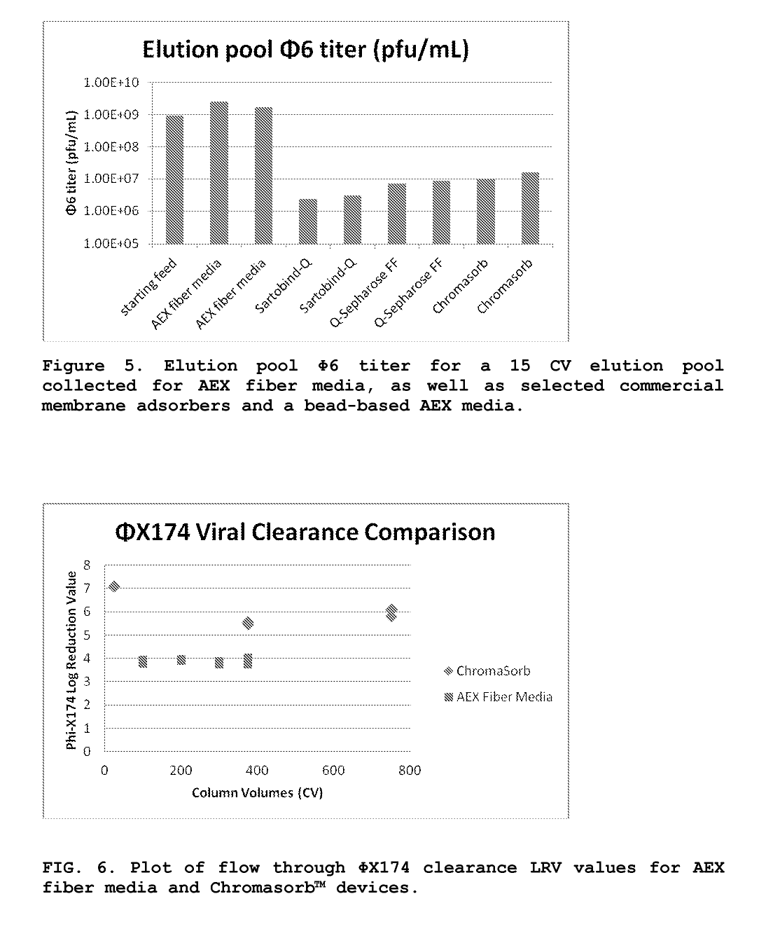

Two 11 mm ID Vantage columns were packed using the AEX Fiber media from Example 2 according to the process described in Example 3. The AEX fiber media columns were attached to a BioCAD chromatography workstation and HETP and peak asymmetry values were measured using a 30 μl injection of 2% acetone solution and 25 mM Tris (pH 8) buffer as eluent at a flow rate of 3.2 mL/min (linear velocity 200 cm/hr). The HETP and peak asymmetry were measured as 0.08 cm and 2.8, respectively. AEX fiber media columns were tested for dynamic binding capacity, viral log reduction value (LRV), and Φ6 recovery using a pseudomonas bacteriophage Φ6 feedstream (1.0×10⁹ pfu/mL in 25 mM Tris pH 8 with 0.0625% HSA) and the performance was compared to that of two commercial anion exchange membrane adsorbers and a commercial bead-based anion exchanger. The AEX Fiber media columns were equilibrated with 35 CV of 25 mM Tris pH 8 with 0.0625% HSA. Afterwards, each column was loaded with 140 CV of a solution of pseudomonas bacteriophage Φ6 feedstream (approximately 9.3×10⁸ pfu/mL in 25 mM Tris pH 8 with 0.0625% HSA) and 20×7 CV flow through fractions were collected. After loading, the columns were washed with 30 CV of 25 mM Tris pH 8 with 0.0625% HSA. The bound Φ6 was eluted with a 15 CV of a 1.0 M NaCl solution in 25 mM Tris pH 8 with 0.0625% HSA. Flow through, wash, and elution samples were analyzed for Φ6 titer by plaque forming assay. The membrane adsorber devices and Q-Sepharose Fast Flow columns were evaluated according to a similar procedure. These devices were equilibrated with 15 CV of 25 mM Tris pH 8 with 0.0625% HSA. Afterwards, each column was loaded with 140 CV of a solution of pseudomonas bacteriophage Φ6 feedstream (approximately 1.4×10⁹ pfu/mL in 25 mM Tris pH 8 with 0.0625% HSA) and 5×28 CV flow through fractions were collected. After loading, the columns were washed with 15 mL of 25 mM Tris pH 8 with 0.0625% HSA. The bound Φ6 was eluted with 15 CV of a 1.0 M NaCl solution in 25 mM Tris pH 8 with 0.0625% HSA. Flow through, wash, and elution samples were analyzed for Φ6 titer by plaque forming assay. The performance data is summarized in Table 4 below and FIGS. 4 and 5. All of the membrane adsorbers (Sartobind-Q and ChromaSorb) as well as the Q-Sepharose Fast Flow resin demonstrated very low binding capacity for Φ6. This is shown by an early breakthrough of Φ6, and the corresponding low Φ6 LRV values reported in the table for the first 28 CV flow through time point. The elution pool Φ6 titers recorded for the membrane adsorber devices and the Q-Sepharose resin column were also quite low, and further reflect the low binding capacity of these materials for the Φ6 bacteriophage. In contrast, for the AEX fiber media columns we find much higher binding capacities for Φ6, with Φ6 LRV of approximately 3 at the same 28 CV flow through time point. The elution pool Φ6 titer is much higher than the comparative samples and the final Φ6 titer is higher than the Φ6 load titer. This indicates that the Φ6 binding capacity for the AEX fiber media columns is substantial and this media is capable of concentrating the Φ6 bacteriophage to values higher than the starting feed.

TABLE 4
Determination of Φ6 LRV and assessment of Φ6 binding
capacity and elution recovery for AEX fiber media, as well as
selected commercial membrane adsorbers and a bead-based AEX media.

| | | | Flow rate, | | | Elution | Φ6 LRV | Elution |
|---|---|---|---|---|---|---|---|---|
| | Column | | mL/min | Load | Φ6 | pool | flow | pool Φ6 |
| | volume | | (residence | volume, | feed | volume, | through | titer |
| Sample | (mL) | | time, min) | CV (mL) | titer | CV | (28 CV) | (pfu/mL) |
| AEX fiber | 2.85 | mL | 2.9 mL/min | 140 CV | 9.3 × 10⁸ | 15 CV | 3.44 | 2.5 × 10⁹ |
| media | | | (1 min) | (400 mL) | | | | |
| AEX fiber | 2.85 | mL | 3.1 mL/min | 140 CV | 9.3 × 10⁸ | 15 CV | 2.88 | 1.7 × 10⁹ |
| media | | | (54 sec) | (400 mL) | | | | |
| Sartobind-Q | 0.14 | mL | 1 mL/min | 140 CV | 1.4 × 10⁹ | 15 CV | 0.18 | 2.4 × 10⁶ |
| | | | (8 sec) | (19.6 mL) | | | | |
| Sartobind-Q | 0.14 | mL | 1 mL/min | 140 CV | 1.4 × 10⁹ | 15 CV | 0.02 | 3.0 × 10⁶ |
| | | | (8 sec) | (19.6 mL) | | | | |
| Q-Sepharose | 1.00 | mL | 1 mL/min | 140 CV | 1.4 × 10⁹ | 15 CV | 0.20 | 7.1 × 10⁶ |
| FF | | | (1 min) | (140 mL) | | | | |
| Q-Sepharose | 1.00 | mL | 1 mL/min | 140 CV | 1.4 × 10⁹ | 15 CV | 0.25 | 8.6 × 10⁶ |
| FF | | | (1 min) | (140 mL) | | | | |
| Chromasorb | 0.08 | mL | 1 mL/min | 140 CV | 1.4 × 10⁹ | 15 CV | 0.21 | 9.8 × 10⁶ |
| | | | (5 sec) | (11.2 mL) | | | | |
| Chromasorb | 0.08 | mL | 1 mL/min | 140 CV | 1.4 × 10⁹ | 15 CV | 0.31 | 1.6 × 10⁷ |
| | | | (5 sec) | (11.2 mL) | | | | |

### Example 7

### Bacteriophage ΦX174 LRV Determination

Two 11 mm ID Vantage columns were packed using the AEX Fiber media from Example 2 according to the process described in Example 3. The AEX fiber media columns were attached to a BioCAD chromatography workstation and HETP and peak asymmetry values were measured using a 30 μl injection of 2% acetone solution and 25 mM Tris (pH 8) buffer as eluent at a flow rate of 3.2 mL/min (linear velocity 200 cm/hr). The HETP and peak asymmetry were measured as 0.10 cm and 2.0, respectively. AEX fiber media columns were tested for viral log reduction value (LRV) using a ΦX174 feedstream (1.28×10⁷ pfu/mL in 25 mM Tris pH 8) and the performance was compared to that of two commercial ChromaSorb™ anion exchange membrane adsorbers. The AEX Fiber media columns were equilibrated with 35 CV (100 mL) of 25 mM Tris pH 8. Afterwards, each column was loaded with 380 CV (1080 mL) of a solution of bacteriophage ΦX174 feedstream (1.28×10⁷ pfu/mL in 25 mM Tris pH 8) and 4×1 mL flow through grab fractions were collected at the 100, 200, 300, and 370 CV time points. The ChromaSorb™ membrane adsorber devices were evaluated according to a similar procedure. These devices were equilibrated with 30 mL (375 CV) of 25 mM Tris pH 8. Afterwards, each device was loaded with 750 CV (60 mL) of a solution of bacteriophage ΦX174 feedstream (approximately 1.28×10⁷ pfu/mL in 25 mM Tris pH 8) and 3×1 mL flow through grab fractions were collected at the 25, 375 and 750 CV time points. The flow through grab samples were analyzed for ΦX174 titer by plaque forming assay. The performance data is summarized in Table 5 below and in FIG. 6. Under these conditions, both the AEX fiber media columns and the ChromaSorb™ membrane adsorber devices demonstrate good ΦX174 viral clearance performance with ΦX174 LRV values greater than or approximately equal to 4.

TABLE 5
Flow through ΦX174 clearance LRV for AEX fiber media
and Chromasorb ™ devices

| | Column | Load | | ΦX174 | ΦX174 |
|---|---|---|---|---|---|
| | volume | volume, | | load titer | LRV |
| Sample ID | (mL) | CV (mL) | | (pfu/mL) | (avg.) |
| AEX Fiber Media | 2.85 mL | 379 | CV | 1.28 × 10⁷ | 3.8 |
| | | (1080 | mL) | | |
| AEX Fiber Media | 2.85 mL | 379 | CV | 1.28 × 10⁷ | 3.9 |
| | | (1080 | mL) | | |
| ChromaSorb ™ | 0.08 mL | 750 | CV | 1.28 × 10⁷ | 6.2 |
| | | (60 | mL) | | |
| ChromaSorb ™ | 0.08 mL | 750 | CV | 1.28 × 10⁷ | 6.2 |
| | | (60 | mL) | | |

### Example 8

### Bind and Elute Purification of Influenza Virus from Clarified MDCK Cell Culture

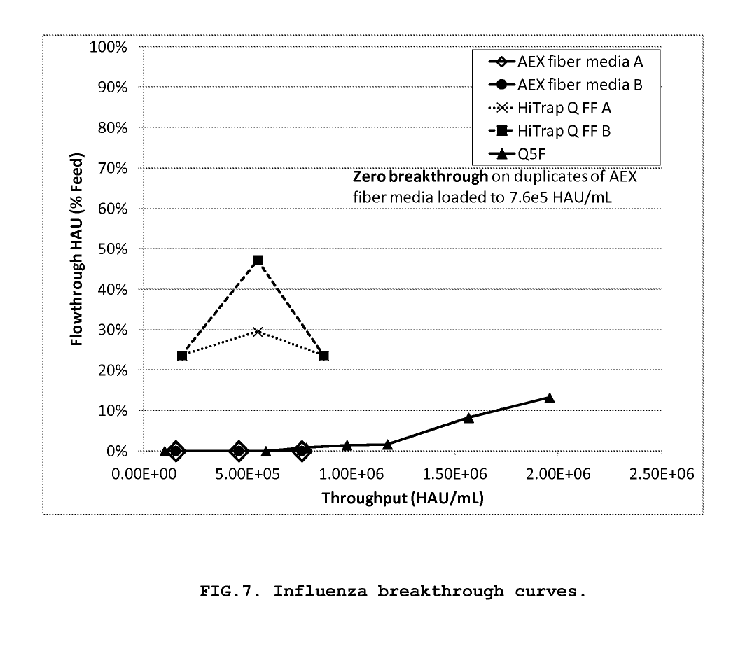

The AEX fiber media from Example 2 was packed into 11 mm Vantage columns according to the procedure described in example 3. The performance of the AEX fiber media was compared with a commercially available AEX bead and a membrane adsorber in the bind/elute purification of influenza virus. Commercial pre-packed Q-type resin HiTrap™ Q FF (GE Healthcare Life Sciences Inc. PN:17-5053-01) as well as a commercial, strongly basic, AEX membrane adsorber device (Sartobind®-Q, Sartorius AG PN:Q5F) were chosen for comparison. Influenza virus cell culture was harvested by settling microcarriers, decantation, and then subsequent filtration through a Stericup®-GP filter unit (EMD Millipore PN:SCGPU11RE) to remove insoluble contaminants. By hemagglutination (HA) assay, the influenza concentration was determined to be 9131 HAU/mL for the starting feed. All devices were equilibrated with at least 5 column volumes (CV) of Sorensen sodium phosphate buffer pH 7.2 with 0.1M NaCl. This same buffer was used for the wash step. Sorensen sodium phosphate buffer pH 7.2 with 1.5M NaCl was used as an elution buffer. Testing was performed on duplicate devices for the AEX fiber media and the HiTrap Q FF devices. The columns were fed using small peristaltic pumps and the membrane device was fed with a 10 mL syringe using slow and steady pressure. Flow-through, load, and elution samples were collected and tested by HA assay. Operating parameters and results are summarized in Tables 6 and 7 below and in FIG. 7. From this evaluation, a low influenza binding capacity is detected for the bead-based HiTrap™ Q FF anion exchanger. This is evidenced by its early influenza breakthrough compared to the Sartobind®-Q membrane adsorber (Q5F). The Sartobind®-Q membrane adsorber demonstrates a higher binding capacity for influenza and upon elution, the bound influenza is recovered with 57% yield. Due to feed limitations, the AEX fiber media devices were only loaded with influenza to 7.6×10⁵ HAU/mL and this material was recovered with a yield of 34 to 67%. Compared to the bead based HiTrap™ Q FF anion exchange media, the AEX fiber media columns demonstrate a much higher binding capacity for influenza and these devices may demonstrate an influenza binding capacity at least as high as the Sartobind®-Q (Q5F) membrane adsorber.

TABLE 6
Operating conditions for B/E influenza purification

| | | LOAD | | |
|---|---|---|---|---|
| | | (# and volume of flow | | |
| | flow rate | through samples | | |
| | (mL/min) | collected) | WASH | ELUTE |
| AEX fiber media | 3.0 | 5 × 50 mL | 15 mL | 15 mL |
| HiTrap Q FF | 1.0 | 5 × 15-20 mL | 4.5 mL | 10 mL |
| Q5F | NA* | 20 × 1.5 mL | 2 mL | 2 mL |
| (Sartobind ®-Q) | | | | |

TABLE 7
Results summary for B/E influenza purification.

| | Load (HAU/mL) | Recovery (%) |
|---|---|---|
| AEX fiber media | 7.6E+05 | 34-67 |
| HiTrap Q FF | 1.8E+05 | 37-61 |
| Q5F | 1.8E+06 | 57 |

### Example 9

### Bind and Elute Purification of Influenza Virus from Clarified MDCK Cell Culture

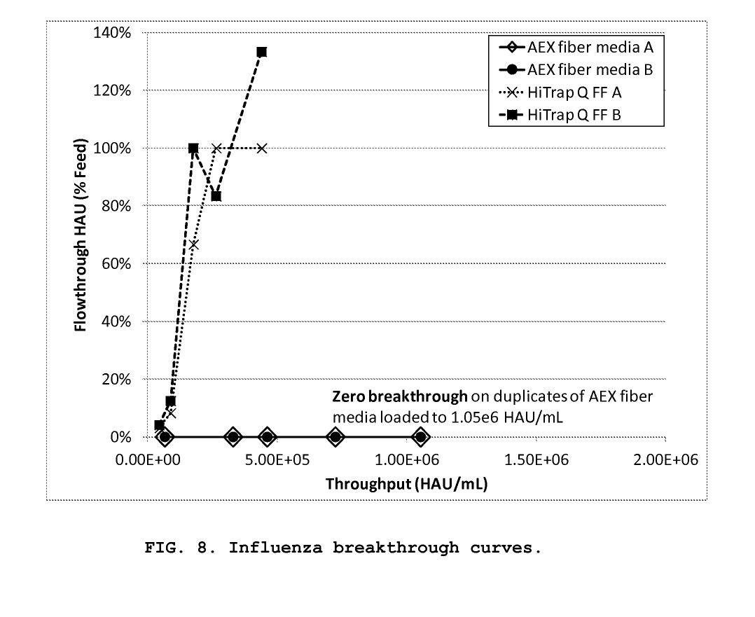

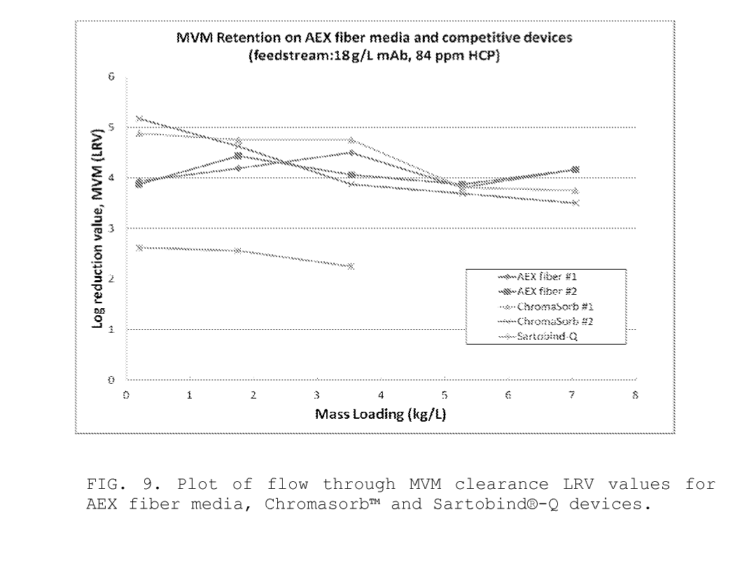

The AEX fiber media from Example 2 was packed into 11 mm Vantage columns according to the procedure described in Example 3. The performance of the AEX fiber media was compared with a commercially available AEX bead in the bind/elute purification of influenza virus. A commercial pre-packed Q-type resin: HiTrap™ Q FF (GE Healthcare Life Sciences Inc. PN:17-5053-01) was chosen for the comparison. Influenza virus cell culture was harvested by settling microcarriers, decantation, and then subsequent filtration through a Stericup®-GP filter unit (EMD Millipore PN:SCGPU11RE) to remove insoluble contaminants. By hemagglutination assay influenza concentration was determined to be 4389 HAU/mL for the starting feed. All devices were equilibrated with at least 5 column volumes (CV) of Sorensen sodium phosphate buffer pH 7.2 with 0.1M NaCl. The same buffer was used for the wash step. Sorensen sodium phosphate buffer pH 7.2 with 1.5M NaCl was used as elution buffer. Testing was done on duplicate devices. The columns were fed using small peristaltic pumps. Flow-through, load and elution samples were collected and tested by HA assay. Operating parameters and results are summarized in Tables 8 and 9 below and in FIG. 8. From this evaluation, a low influenza binding capacity for the bead-based HiTrap™ Q FF anion exchanger is detected. This is evidenced by its early influenza breakthrough compared to the AEX fiber media columns. The AEX fiber media demonstrates a significantly greater binding capacity for influenza and upon elution, the bound influenza is recovered with a 42% yield. Due to feed limitations, the AEX fiber media devices were only loaded with influenza to 1.05×10⁶ HAU/mL and no influenza breakthrough was observed up to this loading level.

TABLE 8
Operating conditions for B/E influenza purification.

| | | LOAD | | |
|---|---|---|---|---|
| | | (# and volume of | | |
| | flow rate | flow through | | |
| | (mL/min) | samples collected) | WASH | ELUTE |
| AEX fiber media | 3.0 | 16 × 45 mL | 30 mL | 15 mL |
| HiTrap Q FF | 1.0 | 10 × 10 mL | 10 mL | 10 mL |

TABLE 9
Results summary for B/E influenza purification.

| | Load (HAU/mL) | Recovery (%) |
|---|---|---|
| AEX fiber media | 1.1E+06 | 42 |
| HiTrap Q FF | 8.8E+04 | 60-100 |

### Example 10

### MVM LRV Determination

Two 6.6 mm ID Omnifit columns were packed using the AEX Fiber media from Example 2 according to the process described in Example 3. For each column, 0.35 g of AEX fiber media was packed to a bed depth of 3.0 cm and a column volume of 1 mL. The viral clearance capability of the AEX fiber media columns were evaluated using a 17.6 g/L mAb feedstream infected with minute virus of mice (MVM) (2.0×10⁶ TCID₅₀/mL) and the performance was compared to that of two commercial ChromaSorb™ devices and one Sartobind-Q anion exchange membrane adsorber. In order to better simulate a relevant mAb feedstream, the feed also contained approximately 84 ppm of host cell protein (HCP) contaminants. The AEX Fiber media columns were equilibrated with 100 CV (100 mL) of 25 mM Tris pH 7. Afterwards, each column was loaded with 411 CV (411 mL) of the MVM-infected mAb feedstream and 5×1 mL flow through grab fractions were collected at the 0.2, 1.8, 3.5, 5.2, and 7.0 kg/L mAb throughput time points. The ChromaSorb™ and Sartobind®-Q membrane adsorber devices were evaluated according to a similar procedure. These devices were equilibrated with 10 mL (125 CV) of 25 mM Tris pH 7. Afterwards, the ChromaSorb™ and Sartobind®-Q devices were loaded with 400 CV (32 mL for ChromaSorb™, 56 mL for Sartobind®-Q device) of the MVM-infected mAb feedstream and 5×1 mL flow through grab fractions were collected at the 0.2, 1.8, 3.5, 5.2, and 7.0 kg/L mAb throughput time points. Note: the 5.2 and 7.0 kg/L mAb throughput time points were not collected for the Sartobind®-Q membrane adsorber device. The flow-through grab samples were analyzed for MVM infection via TCID50 assay. The performance data is summarized in Table 10 below and in FIG. 9. Under these conditions, both the AEX fiber media columns and the ChromaSorb™ membrane adsorber devices demonstrate good MVM viral clearance performance with MVM LRV values 4 at mAb throughput levels as high as 7 kg/L. In contrast, the commercial Sartobind®-Q membrane adsorber device demonstrates a poor MVM LRV value of less than 3, even at a low mAb throughput level.

TABLE 10
Flow through MVM clearance LRV for AEX fiber
media, Chromasorb ™ and Sartobind ®-Q devices

| | | | | MVM/mAb | | |
|---|---|---|---|---|---|---|
| | | Flow rate | | feed | | |
| | | mL/min | | volume, mL | | MVM LRV |
| Sample | CV (mL) | (RT) | | (CV) | | (avg.) |
| AEX Fiber Media | 1.03 | 1.1 | mL/min | 411 | mL | 4.1 |
| | | (54 | sec) | (411 | CV) | |
| AEX Fiber Media | 1.03 | 1.1 | mL/min | 411 | mL | 4.1 |
| | | (54 | sec) | (411 | CV) | |
| ChromaSorb ™ | 0.08 | 1.0 | mL/min | 32 | mL | 4.4 |
| | | (5 | sec) | (400 | CV) | |
| ChromaSorb ™ | 0.08 | 1.0 | mL/min | 32 | mL | 4.2 |
| | | (5 | sec) | (400 | CV) | |
| Sartobind ®-Q | 0.14 | 1.0 | mL/min | 56 | mL | 2.5 |
| | | (8 | sec) | (400 | CV) | |

What is claimed is:

1. A process of purifying a virus in a sample, comprising contacting said sample with a bed of fiber media, the fibers in said media comprising a body region and a plurality of projections extending from said body region, said fibers having imparted thereon functionality enabling chromatography.

2. The process of claim 1, wherein said functionality is grafted to said fibers.

3. The process of claim 1, wherein said functionality enables purification in a flow through mode.

4. The process of claim 1, wherein said functionality enables purification in a bind/elute mode.

5. The process of claim 1, wherein said ion-exchange functionality is cation exchange functionality, and wherein said purification is carried out at a pH ranging from 5 to 8.

6. The process of claim 1, wherein said functionality is anion exchange functionality, and wherein said purification is carried out at a pH ranging from 7 to 9.

7. The process of claim 1, wherein said fibers are functionalized with trimethylamine.

8. The process of claim 1, wherein said virus is influenza.

9. The process of claim 1, wherein fibers have a shape selected from the group consisting of daisy, snowflake and sun shape.
